(19) United States
(12) Patent Application Publication
(10) Pub. No.: US 2011/0300203 A1
(43) Pub. Date: Dec. 8, 2011

(54) CARTILAGE REGENERATION WITHOUT CELL TRANSPLANTATION

(75) Inventor: Jeremy J. Mao, Closter, NJ (US)

(73) Assignee: Trustees of Columbia University in the City of New York, New York, NY (US)

(21) Appl. No.: 13/125,580

(22) PCT Filed: Oct. 22, 2009
(86) PCT No.: PCT/US09/61702

§ 371 Date: Jul. 1, 2011

Related U.S. Application Data
(60) Provisional application No. 61/107,393, filed on Oct. 22, 2008.

(51) Int. Cl. A61K 9/00; A61P 19/02; C12N 5/0775; A61P 19/04; C12N 5/071; A61K 38/18

(52) U.S. Cl. 424/423; 435/395; 435/402; 514/8.9

(57) ABSTRACT

Provided is a method of causing a cell to migrate to a scaffold. Also provided is a method of treating a mammal that has a cartilage defect. Further provided is a tissue scaffold comprising stromal cell-derived factor-1 (SDF-1) and transforming growth factor-β (TGF-β). Additionally, a method of making a tissue scaffold capable of recruiting a cell is provided.

### CROSS-REFERENCE TO RELATED APPLICATION

This application claims the benefit of U.S. Provisional Application No. 61/107,393, filed Oct. 22, 2008, incorporated herein by reference in its entirety.

### STATEMENT REGARDING FEDERALLY SPONSORED RESEARCH OR DEVELOPMENT

This invention was made with government support under Grant No. EB02332, awarded by The National Institutes of Health. The government has certain rights in the invention.

### BACKGROUND

Arthritis is the leading cause of chronic disability, and affects approximately 46 million adults in the U.S. alone. Currently, the predominant approach for cartilage tissue engineering invariably involves cell transplantation (Lammi, 2007). However, cell transplantation for cartilage repair is confronted with critical drawbacks of cell availability, donor site morbidity, and excessive cost associated with cell manipulation. There is thus a need for better methods of cartilage repair. The present invention addresses these problems.

### SUMMARY

The above problems with using cell transplantation for cartilage repair is addressed by the invention described herein, which avoids the use of cell transplantation and instead provides growth factors that encourage cell homing to provide cells for cartilage repair.

In some embodiments, this patent application is directed to a method of causing a cell to migrate to a scaffold. The method comprises placing the scaffold in fluid communication with the cell. In these embodiments, the scaffold comprises stromal cell-derived factor-1 (SDF-1) or a transforming growth factor-β (TGF-β). However, the scaffold does not comprise a transplanted mammalian cell.

The application is also directed to a method of treating a mammal that has a cartilage defect. The method comprises implanting a scaffold at the cartilage defect. In these embodiments, the scaffold comprises stromal cell-derived factor-1 (SDF-1) or a transforming growth factor-β (TGF-β). However, the scaffold does not comprise a transplanted mammalian cell.

Additionally, the application is directed to a tissue scaffold comprising stromal cell-derived factor-1 (SDF-1) and transforming growth factor-β (TGF-β). In these embodiments, the tissue scaffold does not comprise a mammalian cell.

The application is further directed to a method of making a tissue scaffold capable of recruiting a cell. The method comprises combining stromal cell-derived factor-1 (SDF-1) or a transforming growth factor-β (TGF-β) with a scaffold.

### BRIEF DESCRIPTION OF THE DRAWINGS

FIG. 1 is micrographs and a diagram showing scaffold fabrication and in vitro tissue formation. Panel A shows a schematic of a scaffold in accordance with some embodiments of the invention. Panel B shows glistening white tissue formed in a collagen region upon 3 wks cell homing. Panel C shows gelatin microspheres encapsulating cytokines (scale=200 μm).

FIG. 2 is a graph showing growth factor release from gelatin microspheres.

FIG. 3 is micrographs showing DAPI staining for adipose derived stem cells (ASCs) (Panels A-D), bone marrow derived mesenchymal stem cells (MSCs) (Panels E-H), and synovial stem cells (SYNs) (Panels I-L). Panels A, E and I: No cytokines; Panels B, F and J: SDF-1; Panels C, G & K: TGF-β3; Panels D, H and L: SDF-1/TGF-β3. Scale=50 μm.

FIG. 4 is graphs showing cell homing by cytokine delivery. Panel A summarizes experimental results showing that significantly more ASCs were recruited upon 3 wk homing when scaffolds comprised SDF-1+TGF-β3 than scaffolds comprising no growth factor, SDF-1 alone or TGF-β3 alone (n=4-6). Panel B summarizes experimental results showing that significantly more MSCs were recruited upon 3 wk homing when scaffolds comprised TGF-β3 or TGF-β3+SDF1 than scaffolds comprising no growth factor or SDF-1 alone (n=6-8). Panel C summarizes experimental results showing that significantly more SYNs were recruited upon 3 wk homing when scaffolds comprised TGF-β3+SDF1 or SDF1 alone than scaffolds comprising no growth factor or TGF-β3 alone. Open circle: p<0.05 over prior time point; *: significance over other conditions at the same time point.

FIG. 5 shows Alcian blue staining for ASCs (Panels A-D) and MSCs (Panels E-H). Panels A & E: No cytokines; Panels B & F: TGF-β3; Panels C & G: SDF-1; Panels D & H: SDF-1/TGF-β3. Scale=50 μm.

FIG. 6 shows hematoxylin and eosin (H&E) staining for ASCs (Panels A-D) and MSCs (Panels E-H) and SYNs (Panels I-L). Panels A, E and I: No cytokines; Panels B, F and J: SDF-1; Panels C, G & K: TGF-β3; Panels D, H and L: SDF-1/TGF-β3. Scale=50 μm.

FIG. 7 shows toluidine blue staining for ASCs (Panels A-D) and MSCs (Panels E-H) and SYNs (Panels I-L). Panels A, E and I: No cytokines; Panels B, F and J: SDF-1; Panels C, G & K: TGF-β3; Panels D, H and L: SDF-1/TGF-β3. Scale=50 μm.

### DETAILED DESCRIPTION

The inventor has discovered that tissue scaffolds that release the cytokines stromal cell-derived factor-1 (SDF-1) and transforming growth factor-β3 (TGF-β3) recruits surrounding bone marrow derived mesenchymal stem cells (MSCs), adipose derived stem cells (ASCs), and synovial cells (SYNs), which can then differentiate into tissue cells, for example chondrocytes. See Example below. Thus, scaffolds that comprise SDF-1 and a TGF-β are useful for tissue regeneration, particularly cartilage regeneration.

The ability of various cells, including MSCs, to migrate and home to various organs is well-established. See, e.g., Chamberlain et al., 2007; Kan et al., 2005; Loetscher and Moser, 2002; Shi et al., 2007; Sordi et al., 2005; U.S. Patent Application Publication US 2003/0129750 A1; U.S. Patent Application Publication US 2004/0258669 A1; U.S. Patent Application Publication US 2006/0110374 A1; U.S. Patent Application Publication US 2008/0193426 A1; PCT Patent Publication WO 2008/094689 A2.

In some embodiments, methods of causing a cell to migrate to a scaffold are provided. The methods comprise placing the scaffold in fluid communication with the cell. In these embodiments, the scaffold comprises stromal cell-derived factor-1 (SDF-1) or a transforming growth factor-β (TGF-β). However, the scaffold does not comprise a transplanted mammalian cell, i.e., no cell is applied to the scaffold; any cell present in the scaffold migrated into the scaffold.

These methods are not narrowly limited to recruitment of any particular cell type. In particular, the methods are useful for recruiting any undifferentiated or differentiated cells in the MSC lineage, for example osteoblasts, chondrocytes, myocytes, adipocytes, or endothelial cells. In some embodiments, the cell is a precursor cell.

As used herein, a scaffold is in “fluid communication” with a cell if the cell has no physical barrier (e.g., a basement membrane, areolar connective tissue, adipose connective tissue, etc.) preventing the cell from migrating to the scaffold. Without being bound to any particular mechanism, it is believed that the cell migrates to the scaffold along a moist path from its source, in response to the presence of SDF-1 and/or TGF-β forming a concentration gradient to the cell, and thereby influencing the cell to migrate toward the higher concentrations of SDF-1 and/or TGF-β in the scaffold.

As used herein, the singular forms “a”, “an” and “the” are intended to include the plural forms as well, unless the context clearly indicates otherwise. Additionally, the use of “or” is intended to include “and/or”, unless the context clearly indicates otherwise.

The term “cell” as used herein refers to and includes a single cell, a plurality of cells or a population of cells, unless otherwise specified.

As used herein, a “scaffold” is a three-dimensional structure into which cells, tissue, vessels, etc., can grow into, colonize and populate when the scaffold is placed into a tissue site.

As used herein, a “precursor cell”, also known as a progenitor cell, is a cell that is undifferentiated or partially undifferentiated, and can divide and proliferate to produce undifferentiated or partially undifferentiated cells or can differentiate to produce at least one differentiated or specialized cell. Precursor cells include stem cells, including embryonic stem cells and adult stem cells. A precursor cell may be pluripotent, which means that the cell is capable of self-renewal and of trans-differentiation into multiple tissue types upon differentiation. In some embodiments, the precursor cell is a stem cell. In other embodiments, the precursor cell is a chondrogenic precursor cell (i.e., is capable of differentiating into a chondrocyte). Nonlimiting examples of chondrogenic precursor cells include mesenchymal stem cells, adipose-derived stem cells and synovium-derived stem cells.

SDF-1 is a cytokine, the protein product of the SDF1 gene (Bleul et al., 1996); see also GenBank Accession Nos. AJ278857; E09670; E09669; E09668; AF209976; U16752; L12030; L12029; AAH61945; and AAA97434. The last accession provides the human SDF-1 amino acid sequence.

TGF-β is a secreted protein that exists in at least three isoforms, TGF-β1, TGF-β2 and TGF-β3. The peptide structures of the three members of the TGF-β family are highly similar. The mature TGF-β protein dimerizes to produce a 25 KDa active molecule with many conserved structural motifs.

In various embodiments of these methods, the scaffold comprises SDF-1 and a TGF-β. The TGF-β can be, e.g., TGF-β1, TGF-β2 or TGF-β3.

The SDF-1 and/or the TGF-β can have the amino acid sequence of a naturally occurring SDF-1 and/or TGF-β from any mammalian species, including humans. Alternatively, the SDF-1 and/or the TGF-β can have the amino acid sequence modified from a naturally occurring SDF-1 and/or TGF-β provided the SDF-1 and/or TGF-β have substantially the same activity of the naturally occurring protein. The skilled artisan can identify numerous such derivatives of SDF-1 and/or TGF-β without undue experimentation. In some embodiments, the SDF-1 and/or the TGF-β have the amino acid sequence of the human SDF-1 and/or TGF-β.

The SDF-1 and/or TGF-β present in the scaffolds can be at any concentration. In some embodiments, the SDF-1 and/or the TGF-β are present in the scaffold at a concentration of about 10 ng/gram scaffold to about 30,000 ng/gram scaffold. In other embodiments, the SDF-1 and/or the TGF-β are present in the scaffold at a concentration of about 100 ng/gram scaffold to about 3,000 ng/gram scaffold. In additional embodiments, the SDF-1 and/or the TGF-β are present in the scaffold at a concentration of about 200 ng/gram scaffold to about 500 ng/gram scaffold. In still other embodiments, the SDF-1 and/or the TGF-β are present in the scaffold at a concentration of about 300 ng/gram scaffold.

Since different scaffold materials allow release of a given amount of SDF-1 or TGF-β at a different rate, it is also useful to measure the “potency” of the growth factor by how much growth factor is released in a given period of time, e.g., a week. In some embodiments, the SDF-1 and/or TGF-β is released at a rate of about 1 to 1000 ng/gram of scaffold. In other embodiments, the SDF-1 and/or TGF-β is released at a rate of about 10 to 100 ng/gram of scaffold.

These methods are not limited to any particular scaffold size, shape or composition. The scaffold can include, for example, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, or polyacrylate, or a polyethylene oxide-polypropylene glycol block copolymer. The scaffold can be produced from proteins (e.g. extracellular matrix proteins such as fibrin, collagen, and fibronectin), polymers (e.g., polyvinylpyrrolidone), or hyaluronic acid. Synthetic polymers can also be used, including bioabsorbable polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-degradable polymers (e.g., polyacrylates, ethylene-vinyl acetate polymers and other acyl substituted cellulose acetates and derivatives thereof), or non-degradable polyurethanes, polystyrenes, polyvinyl chloride, polyvinyl fluoride, poly(vinylimidazole), chlorosulphonated polyolefins, polyethylene oxide, polyvinyl alcohol, Teflon®, and nylon. Suitable matrix materials are discussed in, for example, Ma and Elisseeff, ed. (2005) Scaffolding in Tissue Engineering, CRC, ISBN 1574445219; Saltzman (2004) Tissue Engineering: Engineering Principles for the Design of Replacement Organs and Tissues, Oxford ISBN 019514130X.

The scaffold can: provide structural and/or functional features of the target tissue (e.g., bone and cartilage of a joint); allow cell attachment and migration; deliver and retain cells and biochemical factors; enable diffusion of cell nutrients and expressed products; and/or exert certain mechanical and biological influences to modify the behavior of the cell phase. The scaffold materials of various embodiments are biocompatible materials that generally form a porous, microcellular scaffold, which provides a physical support for cells migrating thereto.

A scaffold with a high porosity and an adequate pore size is generally preferred so as to facilitate cell introduction and diffusion of cells and nutrients throughout the whole structure. Where the scaffold is bioabsorbable, the rate at which degradation occurs should coincide as much as possible with the rate of tissue formation. Thus, while cells are migrating to the scaffold and fabricating their own natural structure around themselves, the scaffold can provide structural integrity and eventually break down leaving the neotissue, newly formed tissue which can assume the mechanical load. Injectability is also preferred in some clinical applications. In further embodiments, the scaffold can be delivered to a tissue using minimally invasive endoscopic procedures.

The scaffold configuration can be dependent on the tissue or organ that is to be repaired or produced. In some embodiments, the scaffold is a pliable, biocompatible, porous template that allows for target tissue growth. Additionally, the porosity of the scaffold is a consideration that influences cell introduction and/or cell infiltration. See, e.g., PCT Patent Publication WO06004951.

The scaffold is generally fabricated into a shape that follows the geometry of the structure where tissue growth is desired. For example, where cartilage growth on a femoral condyle of a knee joint is desired, the scaffold would be flat and thin. By contrast, where cartilage growth to repair a defect of the nose or ear is desired, the scaffold would take a nose or ear shape.

In some embodiments, the scaffold comprises a microsphere. The microsphere can comprise any material considered to be suitable for tissue engineering. The skilled artisan could identify without undue experimentation a suitable microsphere for any purpose as to material, size, density, or any other physical characteristic. In various embodiments, the microsphere comprises the SDF-1 and/or the TGF-β. In some embodiments, the microsphere comprises a natural polymer, e.g., collagen, gelatin, fibrin, or lysosome. In other embodiments, the microsphere comprises a synthetic polymer, e.g., poly(dl-ε-caprolactone), poly(lactic-coglycolic) acid (PLGA), poly(D,L-lactide) (PLA), poly-L-lactic acid (PLLA), a polyanhydride, or a chitosan, or any of the materials listed above in relation to scaffold composition. The microsphere can also comprise both a natural polymer and a synthetic polymer. In some aspects of these embodiments, the microsphere comprises gelatin cross-linked with glutaraldehyde, as in the Example below.

In some embodiments, the scaffold comprises collagen. In further embodiments, the scaffold comprises calcium alginate. In particular embodiments, the scaffold comprises gelatin microspheres in a layer of cross-linked calcium alginate overlaying a collagen sponge (see Example below).

The microspheres of these embodiments are not narrowly limited to any particular diameter of microsphere. It is envisioned that the most useful size range of microspheres is about 0.002 to about 2,000 μm. In various embodiments, the microsphere diameter is about 0.01 μm, about 0.1 μm, about 1 μm, about 5 μm, about 10 μm, about 25 μm, about 50 μm, about 100 μm, about 250 μm, about 500 μm, about 1000 μm, about 2000 μm, or any size in between these diameters. The microspheres in a scaffold could also have microspheres of varying sizes, encompassing a range between any two of the above diameters.

The method of these embodiments can be practiced in vitro, e.g., as in the Example below. Alternatively, the scaffold is implanted into a living mammal. In some aspects of these embodiments, the SDF-1 and/or the TGF-β has the amino acid sequence of the SDF-1 and/or the TGF-β of the same species as the mammal. In particular embodiments, the mammal is a human.

For in vivo applications, these methods are particularly useful for cartilage repair/replacement. For example, the methods can be used to replace or augment existing cartilage, to introduce new or altered cartilage, to modify artificial prosthesis, or to add to biological tissues or structures.

In some embodiments where the scaffold is implanted into a living mammal, the scaffold is implanted at a site of a cartilage defect in the mammal. The method of these embodiments is not limited to use at any particular cartilage defect site, and could be used in a defect of hyaline cartilage (e.g., at a joint, the nose, the larynx or the sternum), elastic cartilage (e.g., at the ear), or fibrocartilage (e.g., intervertebral discs). Furthermore, the methods are useful for large cartilage defects (i.e., a defect having a surface area greater than about 1 cm²) or smaller defects (e.g., about 1 cm² or smaller, about 0.5 cm² or smaller, about 0.2 cm² or smaller, about 0.1 cm² or smaller, or about 0.05 cm² or smaller).

Cartilage defects in mammals are readily identifiable visually during arthroscopic examination or during open surgery of the joint. Cartilage defects can also be identified inferentially, for example, by using computer aided tomography (CAT scanning), X-ray examination, magnetic resonance imaging (MRI), analysis of synovial fluid or serum markers or by any other procedures known in the art.

Any cartilage defect may be treated using the instant methods, including but not limited to those associated with arthritis (including osteoarthritis and rheumatoid arthritis), osteoporosis, osteochondrosis, osteochondritis, osteogenesis imperfecta, osteomyelitis, osteophytes, achondroplasia, costochondritis, chondroma, chondrosarcoma, herniated disk, Klippel-Feil syndrome, osteitis deformans, osteitis fibrosa cystica, any other congenital defect resulting in absence of a tissue, a tissue defect due to an accident, fracture, wound, joint trauma, an autoimmune disorder, diabetes, cancer, any other disease, disorder, or condition that requires the removal of a tissue, and/or a disease, disorder, or condition that affects the trabecular to cortical bone ratio.

The instant methods are particularly useful for repairing joints, for example the knee, hip, elbow, ankle, or glenohumeral joint. In certain embodiments, the site of the cartilage defect is on a synovial joint condyle. In some aspects of these embodiments, the cartilage defect is due to arthritis, e.g., osteoarthritis or rheumatoid arthritis.

Also provided are methods of treating a mammal that has a cartilage defect. The methods comprise implanting a scaffold at the cartilage defect. In these embodiments, the scaffold comprises stromal cell-derived factor-1 (SDF-1) and/or a transforming growth factor-β (TGF-β). However, the scaffold does not comprise a transplanted mammalian cell.

In some embodiments, the scaffold comprises SDF-1 and a TGF-β. The TGF-β may be, e.g., TGF-β1, TGF-β2 or TGF-β3. As with the methods described above, the SDF-1 and/or the TGF-β can have the amino acid sequence of a naturally occurring SDF-1 and/or TGF-β from any mammalian species, including humans. In some embodiments, the SDF-1 and/or the TGF-β has the amino acid sequence of the SDF-1 and/or the TGF-β of the same species as the mammal.

Any mammal can be treated in these embodiments, including a human.

As with the methods described above, the scaffold for these methods is not limited to any particular size, shape or composition. In some embodiments, the scaffold comprises gelatin microspheres in a layer of cross-linked calcium alginate overlaying a collagen sponge.

Also as with the methods described above, any cartilage defect, from any cause, may be treated using this method. In some embodiments, the site of the cartilage defect is on a synovial joint condyle. In other embodiments, the cartilage defect is due to arthritis.

Additionally, the application is directed to a tissue scaffold comprising stromal cell-derived factor-1 (SDF-1) and transforming growth factor-β (TGF-β). In these embodiments, the tissue scaffold does not comprise a mammalian cell. The TGF-β may be, e.g., TGF-β1, TGF-J32 or TGF-β3.

The SDF-1 and/or the TGF-β can have the amino acid sequence of a naturally occurring SDF-1 and/or TGF-β from any mammalian species, including humans. Alternatively, the SDF-1 and/or the TGF-β can have the amino acid sequence modified from a naturally occurring SDF-1 and/or TGF-β provided the SDF-1 and/or TGF-β have substantially the same activity of the naturally occurring protein. In some embodiments, the SDF-1 and/or the TGF-β have the amino acid sequence of the human SDF-1 and/or TGF-β.

The scaffold of these embodiments can have any size, shape or composition known in the art. In some embodiments, the scaffold comprises a microsphere, for example gelatin cross-linked with glutaraldehyde. In other embodiments, the scaffold comprises collagen. Further, the scaffold can comprise calcium alginate. In particular embodiments, the scaffold comprises gelatin microspheres in a layer of cross-linked calcium alginate overlaying a collagen sponge.

The scaffold can be implanted by any surgical procedure including minimally invasive endoscopic or arthroscopic procedures.

Additionally provided are methods of making a tissue scaffold capable of recruiting a cell, for example a precursor cell. The methods comprise combining stromal cell-derived factor-1 (SDF-1) or a transforming growth factor-β (TGF-β) with a scaffold. In some embodiments, both SDF-1 and a TGF-β is combined with the scaffold. The TGF-β may be, e.g., TGF-β1, TGF-β2 or TGF-β3.

The SDF-1 and/or the TGF-β can have the amino acid sequence of a naturally occurring SDF-1 and/or TGF-β from any mammalian species, including humans. Alternatively, the SDF-1 and/or the TGF-β can have the amino acid sequence modified from a naturally occurring SDF-1 and/or TGF-β provided the SDF-1 and/or TGF-β have substantially the same activity of the naturally occurring protein. In some embodiments, the SDF-1 and/or the TGF-β have the amino acid sequence of the human SDF-1 and/or TGF-β.

The scaffold of these embodiments can have any size, shape or composition known in the art. In some aspects, the scaffold comprises gelatin microspheres that are fabricated from a water-in-oil emulsion then cross-linked with glutaraldehyde. In additional aspects, the microspheres are lyophilized then rehydrated in a solution of the SDF-1 and/or the TGF-β. In further aspects, the scaffold is fabricated with a layer of the microspheres imbedded in cross-linked calcium alginate overlying a collagen sponge.

In some embodiments, the precursor cell is a stem cell. In other embodiments, the precursor cell is a chondrogenic precursor cell. Nonlimiting examples of the chondrogenic precursor cell is a mesenchymal stem cell, an adipose-derived stem cell and a synovium-derived stem cell.

Various embodiments of the application are described in the following Example. Other embodiments within the scope of the claims herein will be apparent to one skilled in the art from consideration of the specification or practice of the application as disclosed herein. It is intended that the specification, together with the Example, be considered exemplary only, with the scope and spirit of the application being indicated by the claims, which follow the example.

### Example 1

### Cartilage Regeneration without Cell Transplantation

### Stem Cell Homing and Concurrent Chondrogenesis In Vitro

This Example describes a study that establishes cartilage regeneration without cell transplantation by 1) homing stem/progenitor cells into an acellular scaffold, and 2) inducing concurrent chondrogenesis of the homed cells. A cytokine delivery system was devised to recruit surrounding stem/progenitor cells and differentiate them into chondrocytes. Prevalent cell types adjacent to an articular cartilage defect include: bone marrow derived mesenchymal stem cells (MSCs), adipose derived stem cells (ASCs) from nearby fat pads, synovial fluid stem cells (SYNs), and native chondrocytes. Of these cell types, ASCs, MSCs and SYNs are studied here for their recruitment by controlled release of cytokines. The data provided herein demonstrate that MSCs, ASCs and SYNs home into acellular scaffolds and concurrently differentiate into chondrogenic cells.

### Methods

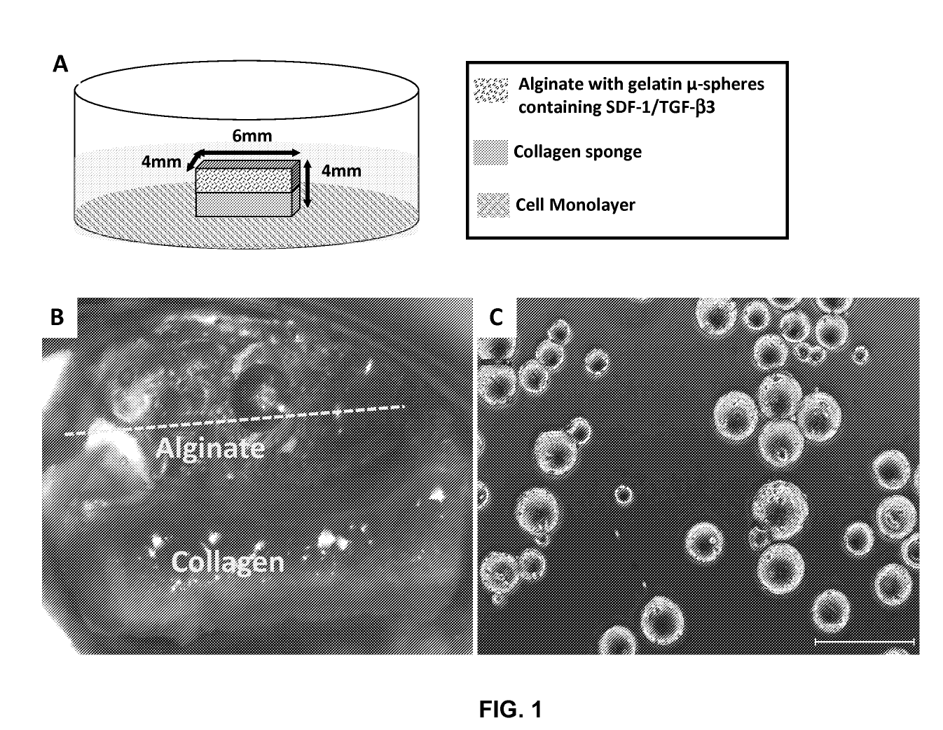

Stromal cell-derived factor-1 (SDF-1) and transforming growth factor-β3 (TGF-β3) were microencapsulated in gelatin microspheres that were fabricated using a water-in-oil emulsion technique and chemically cross-linked with glutaraldehyde (FIG. 1A). Following lyophilization, the microspheres were rehydrated in a solution containing 300 ng/mL TGF-β3, 300 ng/mL SDF-1 or PBS.

Acellular scaffolds were fabricated with two separate but integrated layers consisting of a layer of crosslinked 4% (w/v) calcium alginate containing 30 mg (dry wt) of embedded microspheres and an underlying collagen sponge (FIG. 1B). Human bone marrow MSCs, human ASCs and human SYNs were isolated from adult donors and seeded in 6-well plates (100,000 cells per well). Four conditions were tested: TGF-β3 alone, SDF-1 alone, SDF-1+TGF-β3 and cytokine-free. Each scaffold was placed in the center of the well. Scaffolds were harvested after 3 hours, 1 week and 3 weeks, fixed in 10% formalin, embedded in paraffin and sectioned. DAPI staining was used to assess the number of cells per scaffold section and Alcian Blue to assess chondrogenesis. Hematoxylin and eosin (H&E) staining was done as a confirmation of DAPI and to evaluate the morphology of homed cells. Toluidine blue staining was also utilized to identify proteoglycans and glycosaminoglycans, present in cartilage. Multivariate ANOVA and Bonferroni tests were used for statistical analysis (p<0.05).

### Results

A layer of glistening white tissue was visible in the collagen portion of the scaffolds following 3 wks of cell homing (FIG. 1B). FIG. 1C shows the gelatin microspheres utilized in these studies.

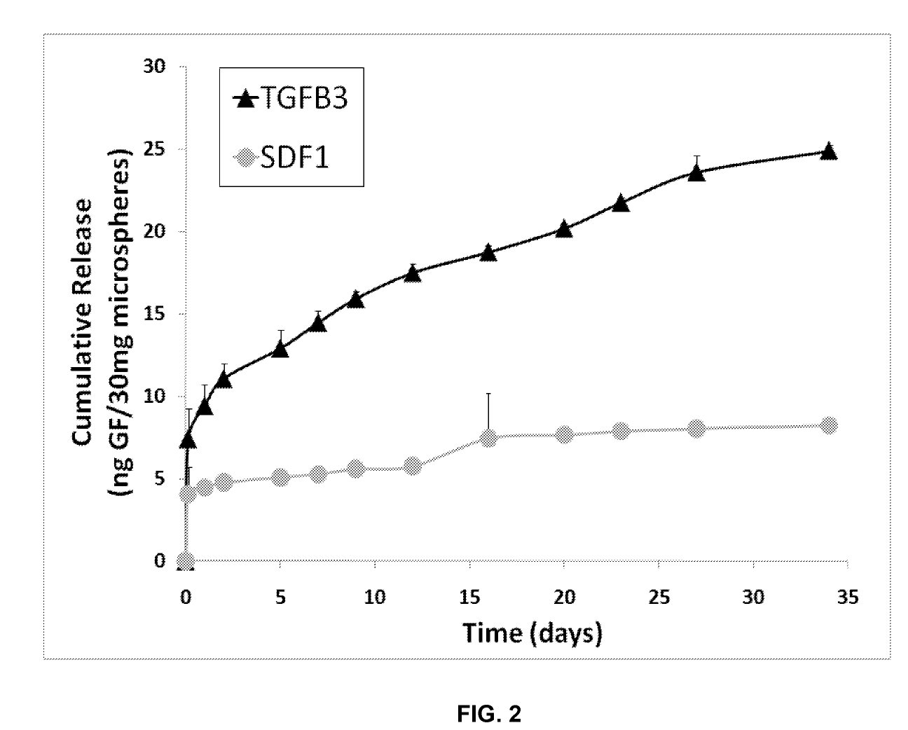

To characterize the growth factor release pattern, gelatin microspheres containing growth factors were cultured in tissue culture dishes containing PBS for 34 days. At select time points, samples of PBS were collected and analyzed using an ELISA assay. As seen in FIG. 2, growth factor was continuously released from the microspheres over 34 days.

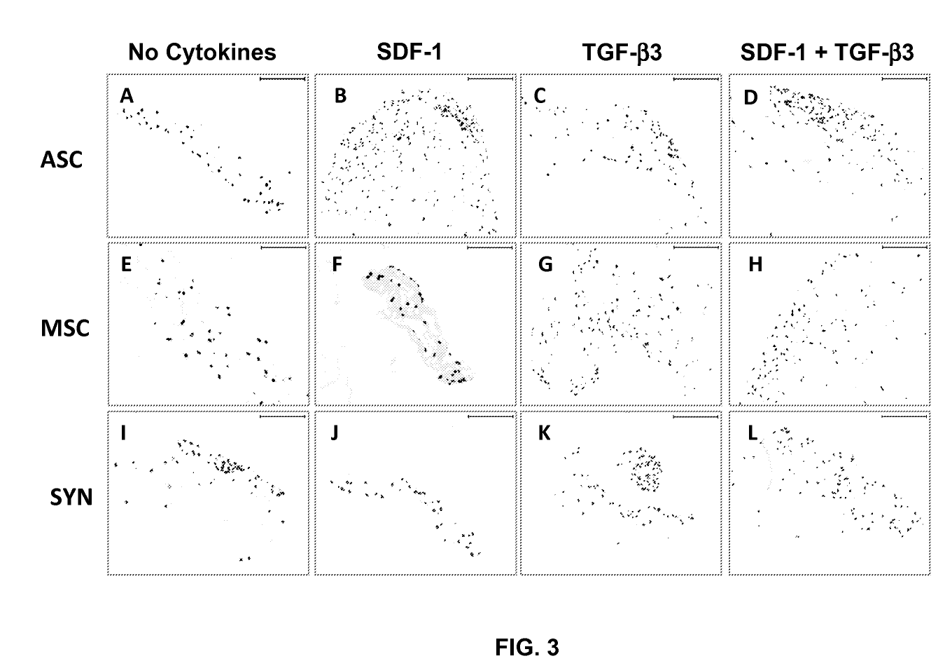

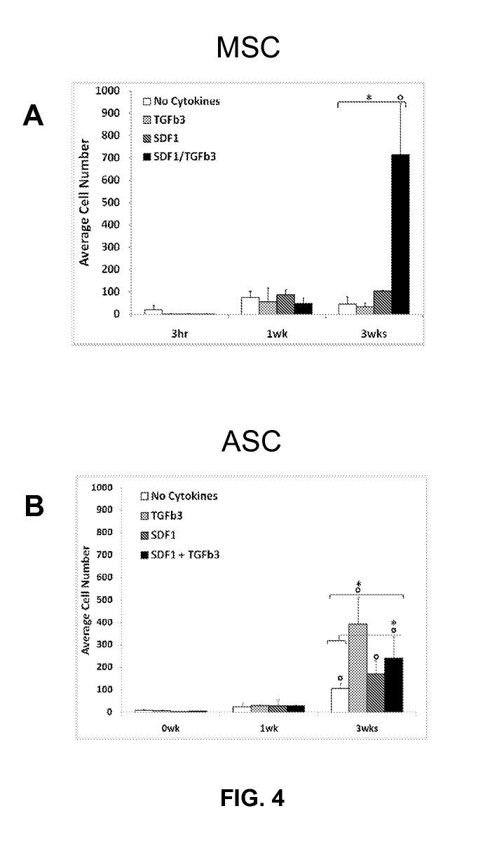

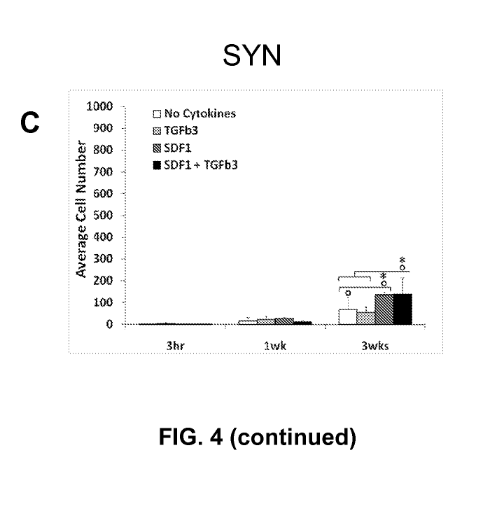

After 6 weeks, scaffolds were fixed, sectioned and stained with DAPI, a cell nucleus stain. DAPI staining confirmed that MSCs, ASCs and SYNs were homed into the collagen scaffold (FIG. 3). DAPI stained scaffolds were quantified to determine the total number of homed cells per scaffold section. After 3 weeks of cell homing, the SDF-1+TGF-β3 group homed the greatest number of MSCs (FIG. 4A). Additionally, TGFIβ3 homed the greatest number of ASCs. Also, both the SDF1 and SDF1+TGFIβ3 groups homed the greatest number of synovial cells.

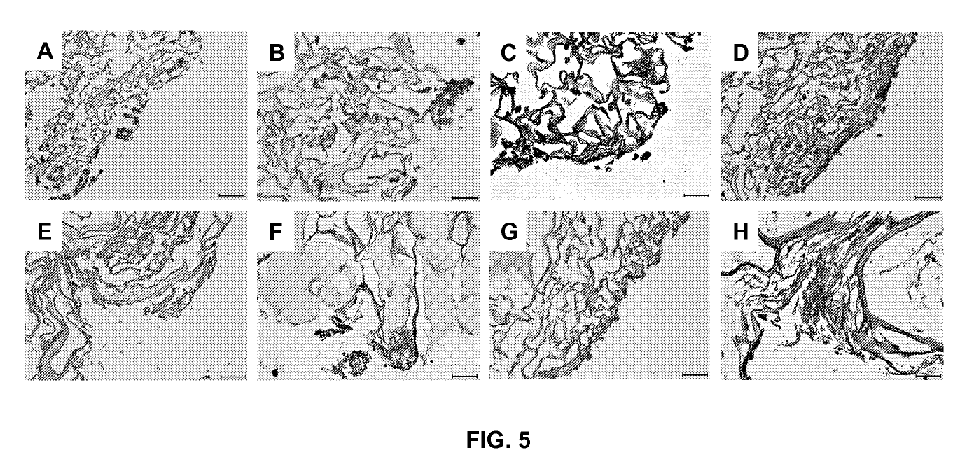

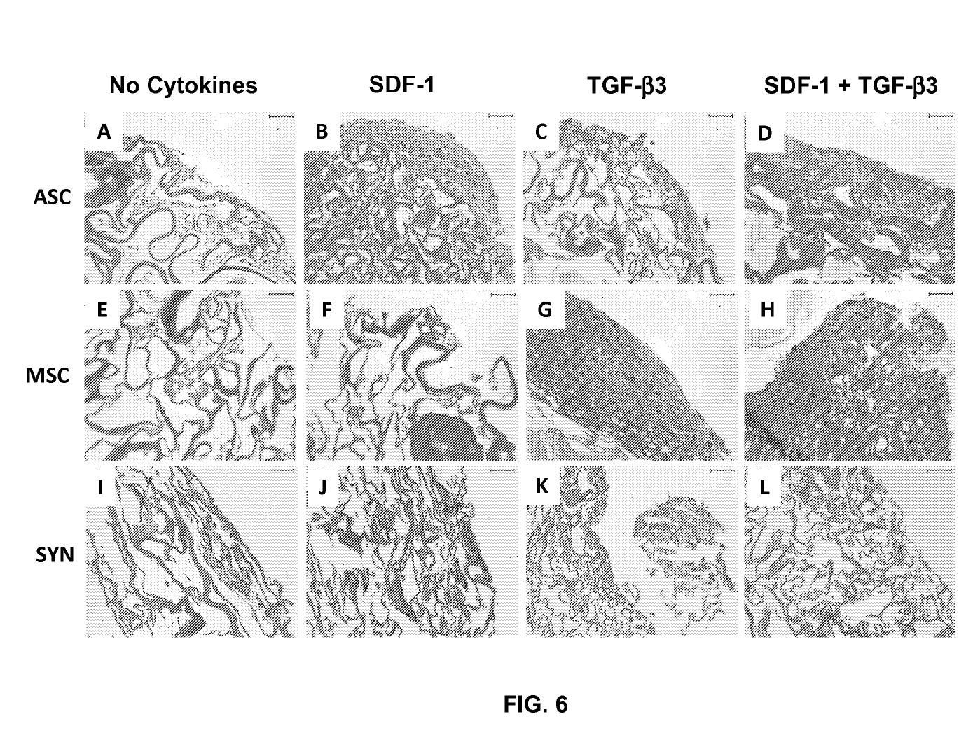

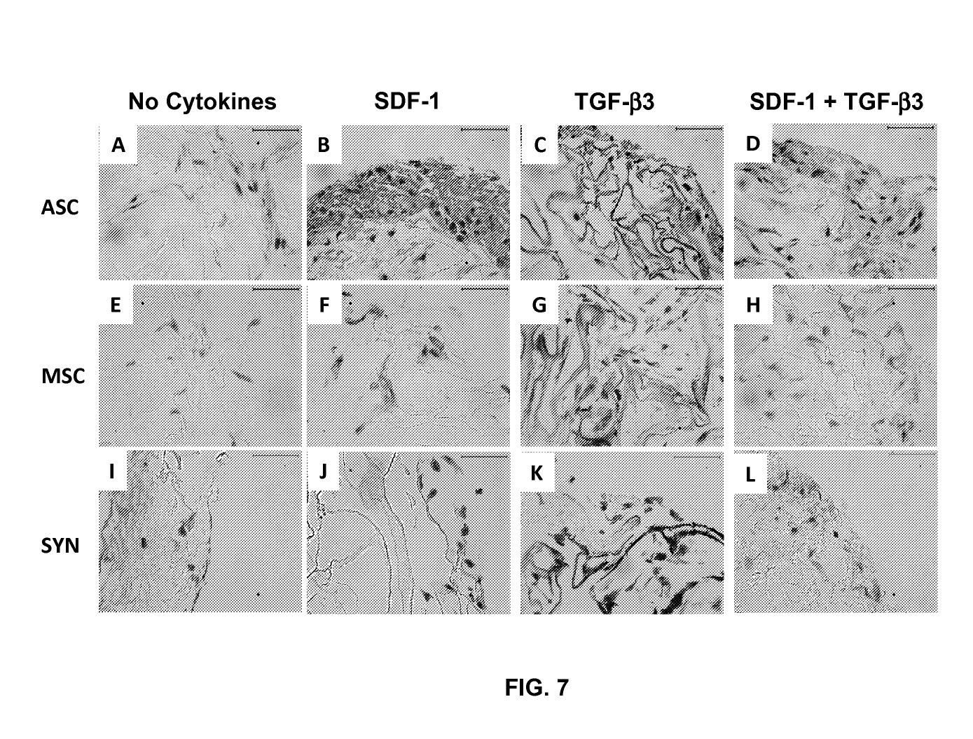

For the ASCs, Alcian blue staining revealed darker staining for TGF-β3 alone and SDF-1+TGF-β3 treatments after 3 wk cell homing (FIG. 5B,D). In contrast, there was minimal blue staining for cytokine-free or SDF-1 alone conditions (FIG. 5A,C). For the MSCs, only the SDF-1/TGF-β3 condition resulted in a significantly greater cell numbers (FIG. 4A) and staining (FIG. 5H) than the other three conditions. These results were confirmed with H&E staining (FIG. 5). The H&E staining also showed increased staining of SYNs in the SDF-1 and SDF-1+TGF-β3 treatments (FIGS. 4C, 6 I-L). Toluidine blue staining indicated the greatest cartilage formation was induced by the TGF-β3 treatment (FIG. 7).

### Discussion

These results demonstrate that this cytokine delivery system not only homes stem/progenitor cells, but also induces chondrogenesis in vitro. Whereas SDF-1 homes ASCs and MSCs, its action alone does not seem to be sufficient for inducing chondrogenesis. In contrast, TGF-β3 shows moderate cell homing effects, but is capable of inducing MSC differentiation into chondrocytes. A combinatory delivery of SDF-1 and TGF-β3 was most effective in homing both ASCs and MSCs, in addition to generating cartilage matrix (as shown by Alcian blue staining). When cultured under the same conditions, homed MSCs show marked chondrogenesis in comparison to ASCs. However, MSCs and ASCs may act synergistically in vivo.

### REFERENCES

Bleul et al., 1996, A Highly Efficacious Lymphocyte Chemoattractant, Stromal Cell-derived Factor 1 (SDF-1), J. Exp. Med. 184:1101-1109.
Buckley, C. D. et al., 2000, J. Immunology 165:3423-3429.
Chamberlain et al., 2007, Stem Cells 25:2739-2749.
Kan et al., 2005, Current Drug Targets 6:31-41.
Lammi, M. J., 2007, Cur. Signal Transduction Ther. 2:41-48.
Loetscher and Moser, 2002, Arthritis Res. 4:233-236.
Ma and Elisseeff, ed., 2005, Scaffolding in Tissue Engineering, CRC, ISBN 1574445219.
Saltzman, 2004, Tissue Engineering: Engineering Principles for the Design of Replacement Organs and Tissues, Oxford ISBN 019514130X.
Shi, M. et al., 2007, Haematologica 92:897-904.
Sordi, V. et al., 2005, Blood 106:419-427.
U.S. Patent Application Publication US 2003/0129750 A1.
U.S. Patent Application Publication US 2004/0258669 A1.
U.S. Patent Application Publication US 2006/0110374 A1.
U.S. Patent Application Publication US 2008/0193426 A1.
PCT Patent Publication WO 2008/094689 A2.
PCT Patent Publication WO 2006/004951 A2.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1-52. (canceled)

53. A method of causing a cell to migrate to a scaffold, the method comprising
placing a scaffold in fluid communication with a cell,
wherein the scaffold comprises stromal cell-derived factor-1 (SDF-1) or a transforming growth factor-β (TGF-β), and
wherein the scaffold does not comprise a transplanted mammalian cell.

54. The method of claim 1, wherein the cell is selected from the group consisting of: a precursor cell, a stem cell, a chondrogenic precursor cell, a mesenchymal stem cell, an adipose-derived stem cell, and a synovium-derived cell.

55. The method of claim 1, wherein the scaffold comprises SDF-1 and a TGF-β.

56. The method of claim 1, wherein the TGF-β is TGF-β3.

57. The method of claim 1, wherein the SDF-1 or the TGF-β have the amino acid sequence of a human SDF-1 or TGF-β.

58. The method of claim 1, wherein the SDF-1 or the TGF-6 are present in the scaffold at a concentration independently selected from the group consisting of: about 10 ng/gram scaffold to about 30,000 ng/gram scaffold; about 100 ng/gram scaffold to about 3,000 ng/gram scaffold; and about 200 ng/gram scaffold to about 500 ng/gram scaffold.

59. The method of claim 1, wherein the scaffold comprises a microsphere, the microsphere comprising the SDF-1 or the TGF-β.

60. The method of claim 59, wherein the microsphere comprises at least one of: gelatin cross-linked with glutaraldehyde; collagen; or calcium alginate.

61. The method of claim 1, wherein the scaffold comprises gelatin microspheres in a layer of cross-linked calcium alginate overlaying a collagen sponge.

62. The method of claim 1, comprising implanting the scaffold into a mammalian subject or a human subject.

63. The method of claim 62, wherein the SDF-1 or the TGF-β has the amino acid sequence of the SDF-1 or the TGF-β of the same species as the subject.

64. The method of claim 62, wherein at least one of the following are satisfied:
(i) the scaffold is implanted at a site of a cartilage defect in the mammal;
(ii) the scaffold is implanted at a site of a cartilage defect of a synovial joint condyle; or
(iii) the scaffold is implanted at a site of an arthritis-associated cartilage defect.

65. The method of claim 64, wherein at least one or more of the following is satisfied:
(i) the cell is selected from the group consisting of: a precursor cell, a stem cell, a chondrogenic precursor cell, a mesenchymal stem cell, an adipose-derived stem cell, and a synovium-derived cell;
(ii) the scaffold comprises SDF-1 and a TGF-β;
(iii) the TGF-β is TGF-β3;
(iv) the SDF-1 or the TGF-β have the amino acid sequence of a human SDF-1 or TGF-β;
(v) the SDF-1 or the TGF-β are present in the scaffold at a concentration independently selected from the group consisting of about 10 ng/gram scaffold to about 30,000 ng/gram scaffold; about 100 ng/gram scaffold to about 3,000 ng/gram scaffold; and about 200 ng/gram scaffold to about 500 ng/gram scaffold;
(vi) the scaffold comprises a microsphere, the microsphere comprising the SDF-1 or the TGF-β;
(vii) the microsphere comprises at least one of gelatin cross-linked with glutaraldehyde; collagen; or calcium alginate;
(viii) the scaffold comprises gelatin microspheres in a layer of cross-linked calcium alginate overlaying a collagen sponge;
(ix) the method comprises implanting the scaffold into a mammalian subject or a human subject;
(x) the SDF-1 or the TGF-β has the amino acid sequence of the SDF-1 or the TGF-β of the same species as the subject;
(xi) the scaffold is implanted at a site of a cartilage defect in the mammal;
(xii) the scaffold is implanted at a site of a cartilage defect of a synovial joint condyle; or
(xiii) the scaffold is implanted at a site of an arthritis-associated cartilage defect.

66. A method of treating a mammalian subject that has a cartilage defect, the method comprising implanting a scaffold at the cartilage defect,
wherein the scaffold comprises stromal cell-derived factor-1 (SDF-1) or a transforming growth factor-β (TGF-β), and
wherein the scaffold does not comprise a transplanted mammalian cell.

67. The method of claim 66, wherein at least one or more of the following is satisfied:
(i) the scaffold comprises SDF-1 and a TGF-β;
(ii) the TGF-β is TGF-β3;
(iii) the SDF-1 or the TGF-β have the amino acid sequence of a human SDF-1 or TGF-β;
(iv) the SDF-1 or the TGF-β has the amino acid sequence of the SDF-1 or the TGF-β of the same species as the subject;
(v) the subject is a human;
(vi) the scaffold comprises gelatin microspheres in a layer of cross-linked calcium alginate overlaying a collagen sponge;
(vii) the site of the cartilage defect is on a synovial joint condyle; or
(viii) the cartilage defect is due to arthritis.

68. A tissue scaffold comprising stromal cell-derived factor-1 (SDF-1) and transforming growth factor-β (TGF-β), wherein the tissue scaffold does not comprise a mammalian cell.

69. The tissue scaffold of claim 67, wherein at least one or more of the following is satisfied:
(i) the TGF-β is TGF-β3;
(ii) the SDF-1 and the TGF-β have the amino acid sequence of a human SDF-1 and TGF-β;
(iii) the scaffold comprises a microsphere;
(iv) the microsphere comprises gelatin cross-linked with glutaraldehyde;
(v) the scaffold comprises collagen;
(vii) the scaffold comprises calcium alginate; or
(viii) the scaffold comprises gelatin microspheres in a layer of cross-linked calcium alginate overlaying a collagen sponge.

70. A method of making a tissue scaffold capable of recruiting a cell, the method comprising combining stromal cell-derived factor-1 (SDF-1) or a transforming growth factor-β (TGF-β) and a scaffold.

71. The method of claim 70, wherein at least one or more of the following is satisfied:
(i) the SDF-1, the TGF-β, and the scaffold are combined;
(ii) the TGF-β is TGF-β3;
(iii) the SDF-1 and the TGF-β have the amino acid sequence of a human SDF-1 and TGF-β;
(iv) the scaffold comprises microspheres, the microspheres comprising the SDF-1 or the TGF-β;
(v) the scaffold comprises gelatin microspheres, the gelatin microspheres comprising the SDF-1 or the TGF-β, and the gelatin microspheres are fabricated from a water-in-oil emulsion then cross-linked with glutaraldehyde;
(vi) the scaffold comprises microspheres, the microspheres comprising the SDF-1 or the TGF-β, and the microspheres are lyophilized then rehydrated in a solution of the SDF-1 or the TGF-β;
(vii) the scaffold is fabricated with a layer of the microspheres imbedded in cross-linked calcium alginate overlying a collagen sponge;
(viii) the scaffold can recruit a cell selected from the group consisting of: a precursor cell, a stem cell, a chondrogenic precursor cell, a mesenchymal stem cell, an adipose-derived stem cell, and a synovium-derived cell.
